(19) United States
(12) Patent Application Publication
(10) Pub. No.: US 2003/0232091 A1
(43) Pub. Date: Dec. 18, 2003

(54) Stabilized retinol for cosmetic dermatological, and pharmaceutical compositions, and use thereof

(76) Inventors: Adi Shefer, East Brunswick, NJ (US); Samuel David Shefer, East Brunswick, NJ (US)

Correspondence Address: Diane Dunn McKay, Esq., Mathews, Collins, Shepherd & McKay, P.A., Suite 306, 100 Thanet Circle, Princeton, NJ, 08540, US

(21) Appl. No.: 10/172,292

(22) Filed: Jun. 17, 2002

(51) Int. Cl. A61K 38/00; A61K 7/42; A61K 7/135; A61K 7/32; A61K 9/14; A61K 9/16; A61K 9/50; A61K 31/07; A61K 31/198; A61K 35/78

(52) U.S. Cl. 424/490; 424/62; 424/725; 424/59; 514/725; 424/65; 514/2; 514/561

(57) ABSTRACT

The present invention relates to a controlled release system useful to stabilize retinol, retinol derivatives, and extracts containing retinol in cosmetic, dermatological, and pharmaceutical compositions. More specifically, the present invention pertains to stabilized retinol in a solid hydrophobic particle that sustains the release of retinol during the product shelf life and enables a gradual and prolonged release of effective levels of retinol and other cosmetic, dermatological, and pharmaceutical active ingredients into biological surfaces. The present invention also refers to a process of stabilizing retinol, retinol derivatives, and extracts containing retinol in cosmetic, dermatological, and pharmaceutical compositions. The invention further relates to cosmetic, dermatological, and pharmaceutical products comprising stable retinol in a hydrophobic particle that can deliver effective levels of retinol and other cosmetic, dermatological, and pharmaceutical active ingredients to biological surfaces over an extended period of time.

### BACKGROUND OF THE INVENTION

1. Field of the Invention

The present invention relates to a controlled release system useful to stabilize retinol, retinol derivatives, and extracts containing retinol in cosmetic, dermatological, and pharmaceutical compositions. More specifically, the present invention pertains to stabilized retinol in a solid hydrophobic particle that sustains the release of retinol during the product shelf life and enables a gradual and prolong release of effective levels of retinol and other cosmetic, dermatological, and pharmaceutical active ingredients onto biological surfaces.

2. Description of the Related Art

Retinol, also known by its common name of vitamin A, is a fat-soluble vitamin composed of a cyclohexene ring with a side chain containing conjugated polyunsaturation. Retinol has the following structure:

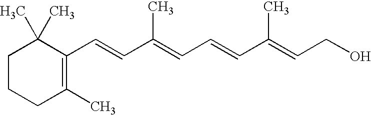

Retinol has long been recognized in the pharmaceutical, nutritional, and cosmetic industry, as an active substance of great benefit. Retinol is essential to many body or biological functions and it plays an important role particularly for vision. The daily requirement is met almost exclusively by the intake of beta-carotene (pro-vitamin A), which is converted into retinol enzymatically.

Because of its important physiological function, especially in the regulation of proliferation and differentiation of a number of cell types, retinol is an extremely valuable substance. In the cosmetics industry, there is great interest in the increased use of retinol, particularly in dermatological formulations. Retinol containing vitamin preparations are being marketed by the food industry. Retinol has also been used in pharmaceutical formulations.

Topical application of retinol stabilizes the vitamin A balance in the skin, which balance can be permanently impaired in particular by exposure to UV light. The deficiency of vitamin A leads to damage, particularly of the epidermis, and to increased formation of wrinkles (“photo-aging”). Deficiency of vitamin A also leads to a loss of the skin's elasticity and weakens the barrier function of the skin against microorganisms.

Retinol has conventionally been used in the treatment of acne as well as repair of skin damage caused either by age or by over-exposure to the sun that retinol has proven to be extremely active. The effects of retinol on cell differentiation make it possible to envisage its use for effectively combating the appearance of wrinkles and fine lines, and for combating dryness, roughness and/or stiffness of the skin. In addition, retinol is active as antioxidants in the regeneration of tissues. Repeated application of cosmetic compositions containing retinol has enabled wrinkles to be removed, the skin to be rendered smooth and small cracks in the epidermis to be repaired.

The main problem that prevents the wide spread use of retinol is its sensitivity to oxidation, in particular oxidation caused by exposure to light. An autoxidation reaction takes place at the side chain of the molecule which contains the conjugated unsaturation. This reaction leads to the formation of numerous decomposition products, to isomerizations and to polymerizations. The originally crystalline retinol material becomes a viscous mass; and the pale yellow color of pure retinol becomes noticeably darker. As a result of peroxides which are formed as intermediates, the potentially toxic results of using these formulations increases. Also, the cosmetically desired results of the remaining intact retinol are reduced.

The term “stable” is defined as a characteristic wherein a composition retains potency for the duration of predetermined expiration period, as defined by generally accepted pharmaceutical protocols, such as “GMP”, or “good manufacturing practices” as promulgated by various trade conventions, such as for example, the United States Pharmacoepia (USP) convention.

Instead of using the uncombined free retinol, which is very sensitive to oxidation, cosmetics, especially anti wrinkle creams, often contain the less effective, but more stable retinyl ester. Examples of these more stable retinyl esters particularly include retinyl acetate and retinyl palmitate.

Long chain retinyl esters, especially retinyl palmitate, have been used extensively in skin cosmetic compositions. Such compositions can be oil-in-water emulsions, as for instance Age Defying Complex™ produced by Chesebrough-Pond's. However, it has been found that retinol or short chain esters of retinol are more efficacious than long chain esters of retinol, such as retinyl palmitate. It is believed that retinyl palmitate does not hydrolyze in-vivo to produce retinol and/or does not penetrate skin.

Retinol and short-chain retinyl esters are more unstable than retinoic acid or long chain retinyl esters, See Idson, “Vitamins and the Skin”, Cosmetics & Toiletries, Vol. 108, December 1993, pp. 79-94, Allured Publishing Corp. (1993); Hoffman-La Roche Inc., Data Sheet “Vitamin A—The ‘Normalizer’”, Roche Vitamins & Fine Chemicals; Hoffman-La Roche Inc., Product Data “Vitamin A Alcohol Blend”. Specifically, they rapidly degrade in the presence of water.

Anhydrous retinol-containing compositions are known in the art. See for instance Dulak et al., U.S. Pat. No. 4,888,363 and Wilmott et al., U.S. Pat. No. 4,826,828. These compositions do not generally include water-soluble skin benefit ingredients, especially if such ingredients are to be included in high amounts.

For these reasons, among others, scientists working in the field have had difficulty in formulating cosmetic, dermatological, and pharmaceutical compositions of stable retinol, which would be useful for cosmetic or dermatological needs. Nevertheless, because of the many beneficial cosmetic, dermatological, and pharmaceutical effects attributed to retinol, numerous attempts have been made to overcome these difficulties.

Reversal of the phases to form water-in-oil emulsions has been proposed as a means to improve stability. Disclosures of emulsions of this type are described in PCT International Publication No. WO 93/00085 (Johnson and Johnson Consumer Products, Inc., publication date Jan. 7, 1993), European Patent Publication No. 440398 (Johnson and Johnson Consumer Products, Inc., publication date Aug. 7, 1991), and European Patent Publication No. 586106 (Johnson and Johnson Consumer Products, Inc., publication date Mar. 9, 1994).

The decomposition problem is addressed to some extent by formulating retinoids to include antioxidants and chelating agents. These have been of limited success, however, particularly in oil-in-water emulsions, which are generally the formulations of choice for skin care products. Oil-in-water emulsions appear to facilitate the diffusion of oxygen to the retinoids, thereby reducing the stabilizing effect of the antioxidants. Water-in-oil emulsions however are greasy and not aesthetically acceptable. In addition, they do not lessen the skin irritation caused by the retinoids.

Many studies have been conducted to stabilize retinol in a cosmetic composition. In particular, there has been proposed an oil-in-water type (O/W type) emulsion wherein retinol is stabilized by an antioxidant such as BHT (butylated hydroxytoluene), BHA (butylated hydroxyanisole), tocopherol and its derivatives, ascorbic acid (vitamin C) and citric acid. For example, U.S. Pat. No. 4,466,805 discloses an O/W type cosmetic composition containing retinol stabilized by an antioxidant such as BHT and dl-.alpha-tocopherol and a chelating reagent such as EDTA (ethylenediaminetetraacetic acid). U.S. Pat. No. 4,247,547 discloses a gel-type cosmetic composition containing retinol stabilized by an antioxidant such as BHT, BHA, ascorbic acid, propyl gallate, and alpha-tocopherol.

However, although retinoids can be stabilized to some extent by these antioxidants, excessive use of antioxidant may cause skin irritation. And, there is a limit in stabilizing retinoids without blocking contact with water which is present in a cosmetic base. Further, since instability of retinoids may be accelerated by oxygen attack through the medium of aqueous solution, many research efforts for stabilization of retinoids have been conducted in a structural aspect of emulsion system itself.

WO 93/00085 describes W/O emulsions comprising retinol and a stabilizing system consisting of a chelating agent such as, for example, EDTA and an antioxidant which may be either a fat-soluble antioxidant such as butylated hydroxytoluene (BHT) or vitamin E, or a water-soluble antioxidant such as vitamin C. According to this disclosure, it is also possible to prepare W/O emulsions containing retinol stabilized by a system consisting of a fat-soluble antioxidant and a water-soluble antioxidant.

EP 0 608 433 describes compositions containing retinol and a stabilizer selected from chelating agents and polysaccharides, oils with an iodine number greater than 70, polyethylene (propylene) glycols, hydroxycarboxylic acid salts, neutral amino acid salts, fat-soluble antioxidants combined with EDTA and with a benzophenone, fat-soluble antioxidants combined with an acidic compound and with a benzophenone, cyclodextrin derivatives in which an antioxidant or a UV-screening agent is included, butanediol and/or fat-soluble antioxidants, water-soluble benzophenone derivatives, basic amino acids and their salts, acidic amino acids and their salts, polar oils and hydrophilic mineral clays.

The use of certain polyamine compounds as antioxidants is known as disclosed in EP 0 209 509.

Avon Products, Inc., the assignee of U.S. Pat. No. 4,826,828, sells two skin care products called Bioadvance and Bioadvance 2000. Each of these products is supplied in two bottles, portions of which are mixed together just prior to use. The first bottle contains what is called “skin lotion”, while the second bottle contains what is called a “fortifier”. The “skin lotion” is a water-in-oil emulsion having a number of ingredients which include water, emulsifiers, silicone and vegetable oils, preservatives, emollients and butylated hydroxytoluene (BHT). The “fortifier” is a solution which contains a number of ingredients including cyclomethicone (a silicone oil), denatured ethanol, an emulsifier (Polysorbate 20), retinol, retinyl acetate, retinyl palmitate, BHT, and BHA. When a specified portion of the “fortifier” is added to a specified portion of the “skin lotion” and mixed, there results a water-in-oil emulsion which comprises retinol, retinyl acetate, retinyl palmitate, BHT and BHA, the latter being oil-soluble antioxidants. The outer package in which Bioadvance is supplied carries a statement of “Because Bioadvance begins to lose effectiveness after one month, for maximum benefits, use a fresh supply each month”. Accordingly, the chemical stability of the retinoids in the mixture of the “skin lotion” and the fortifier” appears to be quite limited. The requirement that both the Bioadvance and Bioadvance 2000 products the “fortifier” ingredients which must be mixed with the “skin lotion” ingredients immediately prior to use indicates that the resulting water-in-oil emulsion which is applied to the skin also has limited chemical stability of one or more of the above-mentioned retinol, retinyl acetate and retinyl palmitate. Thus, the stability of retinol in this type of composition is inadequate for prolonged use. Consequently, frequent and expensive restocking is required.

In WO-A-93/00085, stabilization of retinol in cosmetic compositions, by the addition thereto of a stabilizing complex containing, in association, an antioxidant and a metal ion-chelating agent, is proposed. However, while the stability of retinol appears to be enhanced in such compositions (with 60% of the retinol remaining in the composition after three months storage at 40 degree C.), it is nevertheless true that the relative stability of retinol is due only to the presence of a large quantity of stabilizing chelating agents and antioxidants in the composition.

EP 0 343 444 A2 discloses cosmetic preparations based on retinyl palmitate. Example 3 discloses a night cream in the form of a water-in-oil type emulsion comprising retinyl palmitate and butylated hydroxyanisole (BHA). Example 4 describes a water-in-oil emulsion comprising retinyl acetate and a-tocopherol (Vitamin E).

EP 0 330 496 A2 is directed to skin treatment compositions comprising a topically acceptable base and an effective amount of at least one ester of retinol, said compositions being useful in the treatment of photo-aged skin. Example 6 describes a water-in-oil emulsion comprising vitamin A propionate and BHT, an oil-soluble antioxidant.

U.S. Pat. No. 4,720,353 discloses water-in-oil emulsion carriers for various medicaments and drugs intended for topical application to the skin. Water soluble, miscible or dispersible drugs may be incorporated into the aqueous phase of the emulsion. Oil-soluble, miscible or dispersible drugs may be incorporated into the oil phase. Drugs which may be incorporated into the emulsion include derivatives of retinoic acid. Ingredients which may optionally be added to the emulsion include a preservative such as methyl paraben, propyl paraben or imidazolidinyl urea or an antioxidant such as butylated hydroxyanisole and a water or oil soluble vitamin such as vitamin C, tocopherol linoleate and the like.

U.S. Pat. Nos. 5,559,149 and 5.652,263 describe water-in-oil emulsions which provide a stable retinol formulation. U.S. Ser. Nos. 08/523,836, 08/609,588, and 08/807,351 describe oil-in-water emulsions which provide a stable retinol formulation. U.S. Ser. No. 08/902,922 describes liposome-containing formulations which provide stable retinol formulations.

U.S. Pat. No. 5,738,858 discloses skin care compositions containing fatty hydroxyethyl imidazoline surfactants in combination with retinol and/or retinyl esters for use in stabilizing such compounds.

U.S. Pat. No. 5,756,109 discloses skin care compositions containing geranyl geraniol in combination with retinol and/or retinyl esters for use in stabilizing such compounds.

U.S. Pat. No. 5,759,556 discloses skin care compositions containing cyclic aliphatic unsaturated aldehydes, ketones alcohols or esters in combination with retinol and/or retinyl esters for use in stabilizing such compounds.

U.S. Pat. No. 5,744,148 discloses oil-in-water emulsions containing an unstable retinoid (retinol or ester thereof) in an oil phase. The retinoid is stabilized in the inventive emulsions.

U.S. Pat. No. 5,925,364 discloses a cosmetic or dermatological composition comprising an oil-in-water emulsion comprising oily globules with a lamellar liquid crystal coating. A cosmetic or dermatological composition is described comprising an emulsion of oil-in-water type formed of oily globules which are each provided with a lamellar liquid crystal coating and are dispersed in an aqueous phase. Each oily globule containing at least one lipophilic compound which is cosmetically or dermatologically active is individually coated with a monolamellar or aligolamellar layer obtained from at least one lipophilic surface-active agent, from at least one hydrophilic surface-active agent and from at least one ionic amphiphilic lipid imparting to the emulsion a pH ranging from 5.5 to 7.5, the coated oily globules having a mean diameter of less than 500 nanometers

U.S. Pat. No. 5,980,917 discloses an oil-in-water type cosmetic composition containing retinoids stabilized within a core of a liquid crystal formed by a surfactant having a phase transition temperature of 45 degrees Celsius or higher and a bulky structure.

U.S. Pat. No. 6,015,568 discloses anhydrous stable retinol based cosmetic or pharmaceutical composition for the skin. This composition contains solubilized retinol in an organic solution which is liquid at ambient temperature and which is chosen from aliphatic fatty alcohols having a branched chain at C₁₆-C₂₀, saturated alcoxylated fatty alcohols having a straight or branched chain at C₁₆-C₂₀, the diesters of dicarboxylic esters at C₆-C₁₄ and of isopropyl alcohol, and mixtures of these solvents. This composition is intended for the treatment of skin disorders, and of acne in particular.

U.S. Pat. No. 6,066,328 discloses a method to stabilize active ingredients, including retinol, in cosmetic or dermatological composition comprising an oil-in-water emulsion comprising oily globules with a lamellar liquid crystal coating.

U.S. Pat. No. 6,162,448 discloses a combination of a retinoid with a polyamine polymer. A composition comprising the combination of at least one retinoid selected from the group consisting of vitamin A (retinol) and the bioconvertible precursors of vitamin A and at least one polyamine polymer.

U.S. Pat. No. 6,149,900 discloses a stable W/O/W emulsion and its use as cosmetic and/or dermatological composition. A composition in the form of a water/oil/water triple emulsion comprising an outer aqueous phase and an oily phase constituting, with an inner aqueous phase, a W/O primary emulsion, the outer aqueous phase comprising, in combination, an emulsifying copolymer of carboxylic acid with a fatty chain, and a crosslinked poly(acrylamidomethylpropane-sulfonic acid). The emulsion remains stable, even in the presence of an acidic active agent, and is particularly appropriate as vehicle for water-sensitive and/or oxygen-sensitive active agents, in particular in a cosmetic or dermatological composition. The active agent can be, in particular, a vitamin, such as ascorbic acid or retinol, an enzyme and an alpha or beta-hydroxy acid. The emulsion obtained can constitute, in particular, a composition for cleaning and/or treating and/or protecting the skin and/or mucous membranes and/or keratinous fibers.

U.S. Pat. No. 6,284,234 discloses topical delivery systems for active agents. This invention relates to a method for enhancing the trans-membrane penetration of benefit agents using a certain non-ionic lipid/surfactant-containing formulation as an enhancing agent, and the compositions used therein.

A number of other methods have been adopted to stabilize the breakdown of retinol, such as complexation with cyclodextrins or physical entrapment (absorption) of retinol in polymers. U.S. Pat. No. 2,827,452 discloses stabilizing retinol and also retinyl acetate and retinyl palmitate using beta-cyclodextrin. CA: 98:52237 describes the use of an alpha-cyclodextrin/retinol complex as an additive in food technology. CA: 110:199059 describes increasing the photo-stability of retinyl acetate using beta-cyclodextrin and beta-cyclodextrin derivatives in aqueous solution and in the solid state. CA: 120:265222 discloses the complexation of retinyl acetate using beta-cyclodextrin. WO 94/21225 describes a skincare formulation comprising retinyl palmitate and beta-cyclodextrin. In WO 90/14082, retinoic acid is used together with beta-cyclodextrin in an aqueous gel for dermatological purposes.

U.S. Pat. No. 5,484,816 discloses the stabilization of vitamin A and its corresponding fatty acid esters, which may also be present in dermatological formulations, by using antioxidants and UV-absorbers in the form of cyclodextrin complexes.

J. Drug Targeting 2(5) (1994) 449-54 (CA: 122:64224) reports on the complexation of retinol and retinoic acid using hydroxypropyl-beta-cyclodextrin and on the inclusion of the corresponding complexes in liposomes.

U.S. Pat. No. 5,024,998 describes lowering the risk of undesired accumulation of retinol as a lipophilic pharmaceutical active substance after parenteral application by solubilization using hydroxypropyl-beta-cyclodextrin.

U.S. Pat. No. 5,543,157 discloses an effective amount of active/cyclodextrin complex, in the form of particles having particle sizes below about 12 microns, is incorporated into solid consumer product compositions. The complexes provide fast release of the active when they are wetted even when the amount of water available to effect release of the active is limited as in personal use compositions like drugs, foods, and cosmetics where active release is typically effected by body fluids. Preferred actives include perfumes, flavors, and pharmaceutical materials that are used by consumers.

U.S. Pat. No. 5,985,296 discloses complexes of gamma-cyclodextrin and retinol or retinol derivatives, processes for their preparation and their use. Complexes of gamma-cyclodextrin and retinol or retinol derivatives, along with processes for their preparation and compositions for their use. The complexes are useful in cosmetic formulations and in pharmaceutical formulations.

U.S. Pat. No. 5,851,538 discloses retinoid formulations in porous microspheres for reduced irritation and enhanced stability. Retinoids for topical skin application are formulated as impregnants in porous microspheres. Thus formulated, the retinoids display a surprisingly high level of stability and low degree of skin irritation. The particles are solid, water-insoluble particles, microscopic in size, with a continuous network of pores open to the exterior of the particles, and the particle material is chemically inert with respect to the retinoids and any other ingredients such as chelating agents, antioxidants, and surface-active agents, and further with respect to the conventional ingredients frequently included in aqueous phases of oil-in-water emulsions. The retinoids are not part of the particle matrix, but reside solely in the pores, in which the retinoids are typically deposited by conventional physical means subsequent to the formation of the particles.

Liposome technology was also adopted to stabilize the breakdown of retinol. European Patent application No. EP 472225 describes a pharmaceutical composition based on hydrated lamellar phases or liposomes which contain retinoic acid as the active material, which is said to reduce the irritation while maintaining activity or efficacy.

U.S. Pat. No. 4,911,928 describes another type of lipid vesicle, the paucilamellar vesicles (PLV) which have a capacity of transporting a greater amount of lipophilic material. U.S. Pat. No. 5,147,723, describes non-phospholipid surfactants which can form paucilamellar vesicles.

U.S. Pat. No. 5,679,374 discloses the encapsulation of retinol into two different types of liposome compositions which allow for the simultaneous action of two different active agents, one of which may be retinol and its derivatives. The different liposomes used provide for penetration into different areas of the skin, i.e. surface layers and deep layers.

WO 96/31194 discloses the encapsulation of retinoids into non-phospholipid, non-ionic liposomes which claims for increased chemical stability over a long period of time.

U.S. Pat. No. 5,192,544 discloses the encapsulation of a retinoid compound into phospholipid liposomes with the concomitant incorporation of a pyrimidine derivative which is used to enhance stabilization.

U.S. Pat. No. 5,811,110 discloses fatty acid amides, but not free fatty acids or fatty acid esters, in combination with either retinol or retinyl ester resulted in a synergistic enhancement in keratinocyte proliferation and synergistic inhibition of keratinocyte differentiation. The effects of the retinol or retinyl esters in combination with fatty acid amides were analogous to treatment with retinoic acid.

U.S. Pat. Nos. 5,874,105 and 6,183,774 discloses the encapsulation of vitamin A derivatives in liposomes formulated with long chain alkylammonium fatty acid salts The problems with using liposomes and structured vesicles, as delivery systems, include that these types of systems are very dynamic, unstable in aqueous compositions, and can only be used for encapsulation of certain types of materials (the interior of the liposome is hydrophilic thus will not accommodate retinal which is a lipophilic active ingredient). Stability has become the major problem limiting the use of liposomes for controlled delivery, both in terms of shelf life and after administration.

U.S. Pat. Nos. 5,426,248 and 5,648,091 disclose a process for purifying vitamin A consisting of heating a vitamin A preparation in an inert atmosphere, at a temperature below 170 degrees Celsius and a pressure of less than 4 mm of mercury, said heating taking place in a vessel shielded from light. The vitamin A obtained from this process is mixed with from 0.5:1 to 2:1 parts of tocopherol to vitamin A. The vitamin A and tocopherol mixture can be encapsulated in acacia gum, starch, pectins, lipid hardstocks, ethyl cellulose or other suitable materials. Other antioxidants can be added.

U.S. Pat. No. 5,607,921 discloses a stabilized composition capable of releasing an active agent in contact with the skin, contains at least two precursors of this same active agent, capable of simultaneously releasing this active agent by at least two different specific enzymatic reactions in order to release a large amount of active agent at a faster rate than the sum of the rates of the first enzymatic reaction and of the second enzymatic reaction taken separately, the first precursor being chosen from active agent monosaccharide derivatives and active agent amides. The second precursor is selected from the group consisting of ascorbic acid phosphates, retinol phosphates, tocopherol nicotinates, retinol palmitates, ascorbic acid palmitates, tocopherol acetates, retinol acetates, ascorbic acid acetates, retinol propionates, ascorbic acid propionates, quercetin palmitates, quercetin acetates, quercetin propionates, quercetin ferulates, and mixtures thereof. The composition is useful for dermatological and/or cosmetic treatments applied topically.

U.S. Pat. No. 5,712,311 discloses a cosmetic or dermatological composition with controlled release of active principle containing at least photo-convertible carotenoid, capable of being converted to retinol and retinoic acid or its isomers. This patent also describes the use of a photo-convertible carotenoid for protecting the skin against photo-aging and for preventing acne and to a cosmetic or dermatological composition with controlled release of active principle containing a photo-convertible carotenoid.

Softgel (soft gelatin capsule) formulations have recently become of greater interest in the formulation of products for topical application to the skin, because the softgels provide an attractive single use method for dispensing the product. Typically, the softgels contain 0.1 mL to 2 mL of a fill material, and have a “twist-off” or other removable feature at one end for dispensing the fill material. Softgels can be prepared by methods well known for the preparation of softgels for oral dosage formulations, i.e. by encapsulating the fill material between two sheets of gelatin as it passes between a pair of die rolls having surface cavities shaped to form the desired shape of the resulting softgel.

U.S. Pat. No. 5,587,149 discloses a softgel formulation for water-soluble active ingredients, such as ascorbic acid (vitamin C), where the fill material comprises an emulsion of which a first phase includes polyethylene glycol (into which the water-soluble active ingredient is dissolved) and the second phase includes a silicone fluid.

U.S. Pat. No. 5,891,470 discloses a softgel formulation containing retinol which comprises a soft gelatin shell and a fill material within that shell containing retinol-impregnated microparticles. The fill material may be a optionally thickened silicone oil, or may be an emulsion comprising a silicone oil. Ascorbic acid may be present as ascorbic acid-impregnated microparticles and/or within the emulsion.

U.S. Pat. No. 6,150,422 discloses a stable gelled composition containing lipophilic active agents sensitive to oxygen and/or water or to both, and a solvent which contains, as gelling agent, at least one polysaccharide alkyl ether formed of units containing at least two different glycoside rings, each unit containing at least one hydroxyl group substituted with a saturated hydrocarbon alkyl group.

U.S. Pat. No. 6,228,894 discloses a softgel-compatible composition containing retinol comprises retinol-impregnated microparticles. The composition may include an optionally thickened silicone oil, or may include an emulsion comprising a silicone oil. Ascorbic acid may be present as ascorbic acid-impregnated microparticles and/or within the emulsion. Such compositions are compatible with softgels, and may also be used in other dispensing containers, such as sachets, tubes, and airless pumps. It is well known in the art that unmodified softgels are incompatible with water, and that typical emulsions, whether water-in-oil or oil-in-water, will degrade the gelatin shell of a softgel.

U.S. Pat. No. 5,785,976 discloses suspensions of colloidal solid lipid particles (SLPs) of predominantly anisometrical shape with the lipid matrix being in a stable polymorphic modification and of suspensions of micron and submicron particles of bioactive agents (PBAs); as well as to the use of such suspensions or the lyophilizates thereof as delivery systems primarily for the parenteral administration of preferably poorly water-soluble bioactive substances, particularly drugs, and to their use in cosmetic, food and agricultural products.

U.S. Pat. No. 5,827,520 discloses vehicle and composition containing this vehicle and a stabilized cosmetic or dermatological active substance. A stabilized cosmetic or dermatological active composition containing a vehicle comprising not more than 10% by weight of water, at least one amphiphilic oil, at least one polyol or polyol derivative selected from the group consisting of C₂-C₄ glycols, ether derivatives of a C₂-C₄ glycol and mixtures thereof, and at least one solvent for oil and water, containing an alcohol functional group.

U.S. Pat. No. 5,919,487 discloses relates to nanoparticles, and in particular nanocapsules, provided with a lamellar coating obtained from a silicone surfactant, and to their use in a composition, in particular a topical composition, for treatment of the skin, mucus, nails, scalp and/or hair.

None of the technologies disclosed in prior art of which applicant is aware satisfactorily stabilize retinoids, therefore, it is still desirable to provide an effective process for stabilizing retinol in cosmetic, dermatological and pharmaceutical compositions, which are stable enough at effective retinol levels, to guarantee the results desired in storing and handling these compositions, and enable a gradual and prolong release of retinol and other cosmetic, dermatological, and pharmaceutical active ingredients, which have little or no irritancy and which do not necessitate special ingredients or manufacturing, storage, handling precautions.

### SUMMARY OF THE INVENTION

The present invention relates to a method to stabilize retinol in cosmetic, dermatological, and pharmaceutical compositions. The present invention provides a controlled release system comprising stable retinol which targets biological surfaces of various tissues. The present invention also provides a controlled release system comprising stable retinol which sustains the release of retinol, and enables a gradual and prolonged release of effective levels of retinol and other cosmetic, dermatological, and pharmaceutical active ingredients. Further, the present invention provides retinol in cosmetic, dermatological, and pharmaceutical compositions wherein retinol remains effective for an extended period of time.

The invention provides a controlled release system to stabilize retinol in cosmetic, dermatological, and pharmaceutical compositions, characterized by:

(i) stable retinol in cosmetic, pharmaceutical, and dermatological compositions, over an extended period of time;

(ii) targeted delivery of retinol and other cosmetic, dermatological, and pharmaceutical active ingredient, to biological surfaces comprising the skin, hair, oral cavity, intestine, and biological membranes of various tissues;

(iii) controlled, continuous release of effective levels of retinol and other cosmetic, dermatological, and pharmaceutical active ingredients, over an extended period of time; and

(iv) the release rate of the retinol and other cosmetic, dermatological, and pharmaceutical active ingredient can be synchronized with that of a sensory marker.

The invention also provides a solid hydrophobic particle of encapsulated retinol and other cosmetic, dermatological, and pharmaceutical active agents characterized by:

(i) protection of retinol and other active agents during storage, until needed;

(ii) controlled, continuous release, of effective levels of retinol and other active agents over an extended period of time.

The invention also provides a process for producing the solid particles of the present invention that comprises the steps of:

(i) heating matrix materials, such as solid hydrophobic materials to about 10 degrees C. above the melting point of the hydrophobic materials, with continuous agitation;

(ii) adding retinol and other selected active ingredients to the melt with continuous agitation; and

(iii) cooling said melt to ambient temperature to form a dry free-flowing powder composition.

The present invention provides a composition formed of hydrophobic micro-spheres or particles encapsulating retinol. A molten mixture comprising the hydrophobic material and retinol can be converted into a free-flowing powder by spraying processes known in the art, such as spray chilling, granulation, and the like, to create fine or very fine particles, mostly of a substantially spherical shape, having an average particle diameter of from about 1 micron to about 500 microns, or more preferably having an average particle diameter of from about 0.5 microns to about 50 microns. The particles of the invention can also be produced by drum chilling and grinding.

The invention further relates to cosmetic, dermatological, and pharmaceutical products comprising stable retinol in an hydrophobic particle that can deliver effective levels of retinol and other cosmetic, dermatological, and pharmaceutical active ingredients to biological surfaces over an extended period of time.

The micro-sphere comprising the stable retinol prepared according to the method of this invention can be incorporated into any cosmetic, dermatological, or pharmaceutical compositions known in the art, including liquids, powders, gels, lotions, creams, sprays, sticks, ointments, and pastes. The products can be used for treatment and prevention of sun-induced, photo-aged skin, and related skin disorders. The above-described exemplary cosmetic, dermatological, and pharmaceutical products are preferred in accordance with the present invention, since they permit effective delivery of the retinol into the target biological surface. The introduction of active agents such as retinol and other such active agents targeting biological surfaces comprising the skin and various tissues by sustained release has the advantage of reducing the number of times an active agent must be administered, and further provides a uniform distribution of the active agent over an extended period of time.

In general, the solid hydrophobic particles of the present invention confer several advantages such as high dispersibility in an aqueous medium, and a release rate for the entrapped substance that is controlled by the hydrophobic material barrier properties. These particles also have a lower risk of reaction of substance to be delivered with the vehicle than in emulsion systems because the vehicle is a solid inert material. Moreover, altering the hydrophobic matrix can manipulate the release rate of the substance from the particle. These particles are also easier to prepare than structured vehicles such as liposomes, and are inherently more stable.

### DETAILED DESCRIPTION

The present invention relates to a controlled release system useful to stabilize retinol, retinol derivatives, and extracts containing retinol in cosmetic, dermatological, and pharmaceutical compositions. More specifically, the present invention pertains to stabilized retinol in a hydrophobic micro-sphere or particle that sustains the release of retinol during the product shelf life and enables a gradual and prolong release of effective levels of retinol and other cosmetic, dermatological, and pharmaceutical active ingredients into biological surfaces. The invention further pertains to cosmetic, dermatological, and pharmaceutical compositions, comprising retinol encapsulated in solid hydrophobic particles.

In this disclosure, the term retinol includes retinol, retinol derivatives and extracts containing retinol. For the purposes of the invention, retinol derivatives include retinyl esters (vitamin A esters) and vitamin A acid (retinoic acid).

The terms “cosmetic” or “cosmetic products” or “cosmetic compositions” as used herein, mean (i) articles intended to be rubbed, poured, sprinkled, or sprayed on, introduced into, or otherwise applied to the human or animal body or any part thereof for cleaning, beautifying, promoting attractiveness, or altering the appearance, and (ii) articles intended for use as a component of any such articles, e.g., sun screening compositions, medicinal or first aid creams, and so on.

Retinol can be present in an amount in the range of about 0.01% to about 50% by weight of the composition, and can be stabilized indefinitely in cosmetic, dermatological, and pharmaceutical compositions.

The invention also relates to cosmetic, dermatological, and pharmaceutical products comprising stable retinol that can deliver effective levels of retinol and other cosmetic, dermatological, and pharmaceutical active ingredients to biological surfaces over an extended period of time.

Matrix Materials for Forming the Particles

Suitable solid core materials for forming micro spheres or particles of the present invention are inert nontoxic hydrophobic materials with a melting point range between about 30 degrees C. and about 120 degrees C. Examples of hydrophobic materials include natural, regenerated, or synthetic waxes including: animal waxes such as beeswax, lanolin and shellac wax; vegetable waxes such as carnauba, candelilla, sugar cane, rice bran, and bayberry wax; mineral waxes such as petroleum waxes including paraffin; and microcrystalline wax, ozokrite wax, polyethylene wax, and mixtures thereof. Other hydrophobic materials which can be used in the present invention include wax and silicon copolymers, such as candelilla wax and silicone copolymer, ozokrite wax and silicon copolymers, beeswax and silicon copolymers, and the like. Other hydrophobic compounds which can be used in the present invention include: fatty acid esters such as cetyl palmitate, ethyl stearate, isopropyl myristate, and isopropyl palmitate; high molecular weight fatty alcohols such as cetostearyl alcohol, cetyl alcohol, stearyl alcohol, and oleyl alcohol, solid hydrogenated castor and vegetable oils, hard paraffins, hard fats, and mixtures thereof. Other hydrophobic compounds which can be used, include triglycerides, preferably of at least food grade purity, which can be produced by synthesis or by isolation from natural sources. Natural sources can include animal fat or vegetable oil, such as soy oil, as a source of long chain triglycerides (LCT). Other triglycerides suitable for use in the present invention are composed of a majority of medium length fatty acids (C10-C18), denoted medium chain triglycerides (MCT). The fatty acid moieties of such triglycerides can be unsaturated or polyunsaturated and mixtures of triglycerides having various fatty acid material. Other hydrophobic compounds which can be used in the present invention include synthetic polymers, such as alkylated polyvinylpyrrolidines, the Ganex® copolymer series, and ProLipids® 151, commercially available from the ISP Company. The hydrophobic matrix material can also be a water insoluble silicone based wax, such as the Silwax® wax series, commercially available from Siltech, Inc. of Norcross, Ga. The particle matrix can comprise a single hydrophobic material or a mixture of a plurality of materials. Other hydrophobic materials that are known to those skilled in the art and suitable materials as described in “Industrial Waxes,” Vol. I and II, by Bennett F.A.I.C., published by Chemical Publishing Company Inc., 1975 and Martindale, “The Extra Pharmacopoeia”, The Pharmaceutical Press, 28^{th} Edition pp. 1063-1072, 1982 can be used in the present invention.

Preferred matrix materials are glyceryl monostearate and alkylated polyvinylpyrrolidines, Ganex® V-220 and Ganex® WP-660 copolymer commercially available from the ISP Company.

In another embodiment, the retinol is absorbed on an oil absorbing material prior to incorporating it in the hydrophobic matrix. Examples of such oil absorbing materials are Poly-Pore® E 200 (Allyl Methacrylates Crosspolymer), Poly Pore® L 200, commercially available from Chemdal Corporation, and Silica Shells, commercially available from Kobo Products Inc.

Active Agents

The controlled release system of the present invention includes a primary active agent of retinol compounds selected from retinol, retinol all trans, retinol derivatives, and extracts containing retinol contained in a hydrophobic particle. The hydrophobic particles of the present invention can also include, in addition to retinol compounds, other cosmetic, dermatological, and pharmaceutical active agents, including, but are not limited to: anti-oxidants; free radical scavengers; moisturizers; depigmentation agents; reflectants; humectants; antimicrobial (e.g., antibacterial) agents; allergy inhibitors; anti-acne agents; anti-aging agents; anti-wrinkling agents, antiseptics; analgesics; anti-hair loss agents; hair growth promoting agents; hair growth inhibitor agents; keratolytic agents; anti-inflammatory agents; fresheners; healing agents; anti infectives; inflammation inhibitors; vasoconstrictors; vasodilators; wound healing promoters; peptides, polypeptides and proteins; deodorants and antiperspirants; skin emollients and skin moisturizers; hair conditioners; hair softeners; hair moisturizers; tanning agents; skin lightening agents; antifungals; depilating agents; counterirritants; poison ivy products; poison oak products; burn products; make-up preparations; vitamins; amino acids and their derivatives; herbal extracts; flavoids; sensory markers (i.e., cooling agents, heating agents, etc.); skin conditioners; chelating agents; cell turnover enhancers; coloring agents; sunscreens; nourishing agents; moisture absorbers; sebum absorbers and the like; skin penetration enhancers; and other active ingredients.

Vitamins

Various vitamins can be included in the controlled release system for stabilizing retinol of the present invention. For example, vitamin A and derivatives thereof, vitamin B₂, biotin, pantothenic acid, vitamin K, vitamin D, vitamin E and mixtures thereof can be used.

Antimicrobial and Antifungal Actives

Antimicrobial and antifungal actives can be effective to prevent the proliferation and growth of bacteria and fungi and can be used in the controlled release system for stabilizing retinol of the present invention. Non-limiting examples of antimicrobial and antifungal actives include beta-lactam drugs, quinolone drugs, ciprofloxacin, norfloxacin, tetracycline, erythromycin, amikacin, 2,4,4′-trichloro-2′-hydroxy diphenyl ether, 3,4,4′-trichlorobanilide, phenoxyethanol, phenoxy propanol, phenoxyisopropanol, doxycycline, capreomycin, chlorhexidine, chlortetracycline, oxytetracycline, clindamycin, ethambutol, hexamidine isethionate, metronidazole, pentarnidine, gentamicin, kanamycin, lineomycin, methacycline, methenamine, minocycline, neomycin, netilmicin, paromomycin, streptomycin, tobramycin, miconazole, tetracycline hydrochloride, erythromycin, zinc erythromycin, erythromycin estolate, erythromycin stearate, amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, amanfadine hydrochloride, arnanfadine sulfate, octopirox, parachlorometa xylenol, nystatin, tolnaftate, zinc pyrithione; clotrimazole; alantolactone; isoalantolactone; alkanet extract (alaninin); anise; arnica extract (helenalin acetate and 11, 13 dihydrohelenalin); Aspidium extract (phloro, lucinol containing extract); barberry extract (berberine chloride); bay sweet extract; bayberry bark extract (myricitrin); benzalkonium chloride; benzethonium chloride; benzoic acid and its salts; benzoin; benzyl alcohol; blessed ! 5 thistle; bletilla tuber; bloodroot; bois de rose oil; burdock; butyl paraben; cade oil; CAE (available from Ajinomoto, located in Teaneck, N.J.); cajeput oil; Cangzhu; capsicum frutescens extract; caraway oil; cascarilla bark (sold under the tradename ESSENTIAL OIL); cedarleaf oil; chamomille; chaparral; chlorhexidine gluconate; chlorophenesin; chlorxylenol; cinnamon oil; citronella oil; clove oil; Crinipan AD (available from Climbazole); 2,3-dihydro-farnesol; dehydroacetic acid and its salts; dill seed oil; DOWICIL 200 (available from Dow Chemical, located in Midland, Mich.); echinacea; elenolic acid; epimedium; ethyl paraben; Fo-Ti; galbanum; garden bumet; GERMALL 115 and GERMALL II (available from ISP-Sutton Labs, located in Wayne, N.J.); German chamomile oil; giant knotweed; GLYDANT (available from Lonza, located in Fairlawn, N.J.); GLYDANT PLUS (available from Lonza); grapefruit seed oil; 1,6 hexanediol; hexamidine diisethionate; hinokitiol; honey; honeysuckle flower; hops; immortelle; iodopropynl butyl carbamide (available from Lonza); isobutyl paraben; isopropyl paraben; JM ACTICARE (available from Microbial Systems International, located in Nottingham, NG); juniper berries; KATHON CG (available from Rohm and Haas, located in Philadelphia, Pa.); kojic acid; labdanum; lavender; lemon balm oil; lemon grass; methyl paraben; mint; mume; mustard; myrrh; neem seed oil; ortho phenyl phenol; olive leaf extract (available from Bio Botanica); parsley; patchouly oil; peony root; 1,2 pentandiol; PHENONIP (available from Nipa Labs, located in Wilmington, Del.); phenoxyethanol; phytosphingosine; pine needle oil; PLANSERVATIVE (available from Campo Research); propyl paraben; purslane; quillaira; rhubarb; rose geranium oil; rosemary; sage; salicylic acid; sassafras; savory; sichuan lovage; sodium meta bisulfite; sodium sulfite; SOPHOLIANCE (available from Soliance, located in Compiegne, France); sorbic acid and its salts; sphingosine; stevia; storax; sucrose esters; tarmic acid; tea; tea tree oil (cajeput oil); thyme; triclosan; triclocarban; tropolone; turpentine; umbelliferone (antifungal); yucca; and mixtures thereof.

Anti-Inflammatory Agents

Anti-inflammatories can be included in the controlled release system for stabilizing retinol of the present invention to enhance photoprotection benefits, particularly from UVA. Suitable steroidal anti-inflammatories include hydrocortisone; non-steroidal anti-inflammatories such as oxicans, salicylates, acetic acid derivatives, fenamates, propionic acid derivatives, pyrazoles, substituted phenyl compounds, 2-naphthyl containing compounds, and natural anti-inflammatories such as aloe vera. Examples of anti-inflammatories are described in U.S. Pat. No. 5,487,884, the entire contents of which are incorporated herein by reference.

Anti-Acne Agents

Anti-acne agents can be included in the controlled release system for stabilizing retinol of the present invention. Non-limiting examples of useful anti-acne actives include the keratolytics such as salicylic acid (o-hydroxybenzoic acid), derivatives of salicylic acid such as 5-octanoyl salicylic acid and 4 methoxysalicylic acid, and resorcinol; retinoids such as retinoic acid and its derivatives (e.g., cis and trans); sulfur-containing D and L amino acids and their derivatives and salts, particularly their N-acetyl derivatives, a preferred example of which is N-acetyl-L-cysteine; lipoic acid; antibiotics and antimicrobials such as benzoyl peroxide, octopirox, tetracycline, 2,4,4′-trichloro-2′-hydroxy diphenyl ether, 3,4,4′-trichlorobanilide, azelaic acid and its derivatives, phenoxyethanol, phenoxypropanol, phenoxyisopropanol, ethyl acetate, clindamycin and meclocycline; sebostats such as flavonoids and bioflavonoids; bile salts such as scymnol sulfate and its derivatives, deoxycholate, and cholate; abietic acid; adapalene; allantoin; aloe extracts; arbietic acid and its salts; aryl-2,4 dioxo oxazolidine derivatives; ASEBIOL (available from Laboratories Serobiologiques, located in Somerville, N.J.); azaleic acid; barberry extracts; bearberry extracts; belancanda chinensis; benzoquinolinones; benzoyl peroxide; berberine; BIODERMINE (available from Sederma, located in Brooklyn, N.Y.); bioflavinoids; bisabolol; S-carboxymethyl cysteine; carrot extracts; cassin oil; clove extracts; citral; citronellal; climazole; Completech MBAC-OS (available from Lipo); CREMOGEN M82 (available from Dragoco, located in Totowa, N.J.); cucumber extracts; dehydroacetic acid and its salts; dehydroeplandersterone salicylate; dichlorophenyl imidazoldioxolan which is commercially available as COMPLETECH MBAC-OS (from Lipo, located in Paterson, N.J.); DL valine and its esters; DMDM hydantoin; Epicutin TT (available from CLR); erythromycin; escinol; ethyl hexyl monoglyceryl ether; ethyl 2-hydroxy undecanoate; farnesol; famesol acetate; geranoil; glabridin; gluconic acid; gluconolactone; glyceryl monocaprate; glycolic acid; grapefruit seed extract; gugu lipid; Hederagenin (available from Maruzen); hesperitin; hinokitol; hops extract; hydrogenated rosin; 10 hydroxy decanoic acid; ichtyhol; interleukin 1 alpha antagonists; iodo-2-propynyl butyl carbamate; Kapilarine (available from Greentech); ketoconazole; lactic acid; lemon grass oil; Lichochalcone LR15 (available from Maruzen); linoleic acid; LIPACIDE C8CO (available from Seppic, located in Paris, France); lovastatin; 4 methoxysalicylic acid; metronidazole; minocycline; mukurossi; neem seed oil; vitamin B.sub.3 compounds (such as niacinamide and nicotinic acid); nisin; 5-octanoly salicylic acid; octopirox; panthenol; 1-pentadecanol; peonia extract; peppermint extract; phelladendron extract; 2-phenyl-benzothiophene derivatives; phloretin; PHLOROGINE (available from Secma); phosphatidyl choline; proteolytic enzymes; quercetin; red sandalwood extract; resorcinol; rosemary extract; rutin; sage extract; salicin; salicylic acid; skull cap extract; siber hegner extract; siberian saxifrage extract; silicol; sodium lauryl sulfate; sodium sulfoacetamide; Sophora Extract (available from Maruzen); sorbic acid; sulfur; sunder vati extract; tea tree oil; tetracyline; tetra hydroabietic acid; thyme extract; tioxolone; tocopherol; trehalose 6-undecylenoate; 3 tridecene-2-ol; triclosan; tropolone; UNITRIENOL T27 (available from Unichem, located in Gouda, Netherlands); vitamin D₃ and its analogs; white thyme oil; willow bark extract; wogonin; Ylang Ylang; zinc glycerolate; zinc linoleate; zinc oxide; zinc pyrithione; zinc sulfate and mixtures thereof

Anti-Wrinkle, Anti-Skin Atrophy and Skin Repair Actives

Anti-wrinkle, anti-skin atrophy and skin repair actives can be effective in replenishing or rejuvenating the epidermal layer and can be included in the controlled release system for stabilizing retinol of the present invention. These actives generally provide these desirable skin care benefits by promoting or maintaining the natural process of desquamation. Nonlimiting examples of anti-wrinkle and anti-skin atrophy actives include vitamin B3 compounds (such as niacinamide and nicotinic acid), salicylic acid and derivatives thereof (such as 5-octanoyl salicylic acid, heptyloxy 4 salicylic acid, and 4-methoxy salicylic acid); sulfur-containing D and L amino acids and their derivatives and salts, particularly the N-acetyl derivatives, a preferred example of which is N-acetyl-L-cysteine; thiols, e.g. ethane thiol; hydroxy acids, phytic acid, lipoic acid; lysophosphatidic acid; skin peel agents (e.g., phenol and the like); Actein 27-Deoxyactein Cimicifugoside (available from Cirnigoside); adapalene; ademethionine; adenosine; aletris extract; alkyl glutathione esters; alkoxyalkoxy alkoxyn benzoic and derivatives; aloe derived lectins; amino propane phosphoric acid; 3-aminopropyl dihydrogen phosphate; Amadorine (available from Barnet Products); anise extracts; AOSINE (available from Secma); arginine amino benzoate; ASC III (available from E. Merck, located in Darmstadt, Germany); ascorbic acid; ascorbyl palmitate; asiatic acid; asiaticosides; ARLAMOL GEO™ (available from ICI, located in Wilmington, Del.); azaleic acid; benzoic acid derivatives; bertholletia extracts; betulinic acid; BIOCHANIN A AND BIOPEPTIDE CL (available from Sederma, located in Brooklyn, N.Y.); BIOPEPTIDE EL (available from Sederma); biotin; blackberry bark extract; blackberry lily extracts; black cohosh extract; blue cohesh extract; butanoyl betulinic acid; carboxymethyl 1,3 beta glucan; catecholamines; chalcones; citric acid esters; chaste tree extract; clover extracts; coumestrol; CPC Peptide (available from Barnet Products); daidzein; dang gui extract; darutoside; debromo laurinterol; 1-decanoyl-glycero-phosphonic acid; dehydrocholesterol; dehydrodicreosol; dehydrodieugenol; dehydroepiandersterone; DERMOLECTINE (available from Sederma); dehydroascorbic acid; dehydroepiandersterone sulfate; dianethole; dihydroxy benzoic acid; 2,4 dihydroxybenzoic acid; diglycol guanidine succinate; diosgenin; disodium ascorbyl phosphate; dodecanedioic acid; Ederline (available from Seporga); Enderline (available from Laboratories Seporga); equol; eriodictyol; estrogen and its derivatives; ETF (available from Laboratories Seporga); ethocyn; ELESERYL SH (available from Laboratories Serobiologiques, located in Somerville, N.J.); ENDONUCLEINE (available from Laboratories Serobiologiques); ergosterol; eythrobic acid; fennel extract; fenugreek seed extract; FIBRASTIL (available from Sederma); FIBROSTIMULINES S and P (available from Sederma); FIRMOGEN LS 8445 (available from Laboratories Serobiologiques); formononetin; forsythia fruit extract; gallic acid esters; gamma amino butyric acid; GATULINE RC (available from Gattlefosse, located in Priest, France); genistein; genisteine; genistic acid; gentisyl alcohol; gingko bilboa extracts; ginseng extracts; ginsenoside (RO, R₆₋₁, R₆₋₂, R₆₋₃, R_{C}, R_{D}, R_{E}, R_{F}, R_{F-2}, R_{G-1}, R_{G-2}); gluco pyranosyl-L-ascorbate; glutathione and its esters; glycitein; hesperitin; hexahydro curcumin; HMG-coenzyme A reductase inhibitors; hops extracts; 11 hydroxy undecanoic acid; 10 hydroxy decanoic acid; 25-hydroxycholesterol; 7-hydroxylated sterols; hydroxyethyl isostearyloxy isopropanolamine; hydroxy-tetra methyl piperidinyloxy; hypotaurine; ibukijakou extract; isoflavone SG 10 (available from Barnet Products); kinetin; kohki extract; L-2-OXO-thiazolidine-4-carboxylic acid esters; lactate dehydrogenase inhibitors; 1-lauryl, -lyso-phosphatidyl choline; lectins; lichochalcone LF15 (available from Maruzen); licorice extracts; lignan; lumisterol; lupenes; luteolin; lysophosphitidic acid; magnesium ascorbyl phosphate; margin; melatonin; melibiose; metalloproteinase inhibitors; methoprene; methoprenic acid; mevalonic acid; MPC COMPLEX (available from CLR); N methyl serine; N methyl taurine; N,N¹-bis (lactyl) cysteamine; naringenin; neotigogenin; o-desmethylangoiensin; oat beta glucan; oleanolic acid; pantethine; phenylalanine; photoanethone; piperdine; placental extracts; pratensein; pregnenolone; pregnenolone acetate; pregnenolone succinate; premarin; quillaic acid; raloxifene; REPAIR FACTOR 1 and REPAIR FACTOR FCP (both available from Sederma); retinoates (esters of C₂-C₂₀ alcohols); retinyl glucuronate; retinyl linoleate; S-carboxymethyl cysteine; SEANAMINE FP (available from Laboratories Serobiologiques); sodium ascorbyl phosphate; soya extracts; spleen extracts; tachysterol; taurine; tazarotene; tempol; thymulen; thymus extracts; thyroid hormones; tigogenin; tocopheryl retinoate; toxifolin; traumatic acid; tricholine citrate; trifoside; uracil derivatives; ursolic acid; vitamin D₃ and its analogs; vitamin K; vitex extract; yam extract; yamogenin; zeatin; and mixtures thereof.

Skin Barrier Repair Actives

Skin barrier repair actives are those skin care actives which can help repair and replenish the natural moisture barrier function of the epidermis and can be included in the controlled release system for stabilizing retinol of the present invention. Non-limiting examples of skin barrier repair actives include Alpha Lipid (available from Lucas Meyer); ascorbic acid; biotin; biotin esters; brassicasterol; caffeine; campesterol; canola derived sterols; Cennamides (available from Ennagram); Ceramax (available from Alban Muller); CERAMAX (available from Quest, located in Ashford, England); CERAMIDE 2 and CERAMIDE HO3™ (both available from Sederma); CERAMIDE II (available from Quest); CERAMIDE III and IIIB (both available from Cosmoferm, located in Deft, Netherlands); CERAMIDE LS 3773 (available from Laboratories Serobiologiques); CERAMINOL (available from Inocosm); Cerasol and Cephalip (both available from Pentapharm); cholesterol; cholesterol hydroxystearate; cholesterol isostearate; 7 dehydrocholesterol; DERMATEIN BRC and DERMATEIN GSL (both available from Hormel); ELDEW CL 301 AND ELDEW PS 203 (both available from Ajinomoto); Fitobroside (available from Pentapharm); galactocerebrosides; Generol 122 (available from Henkel); glyceryl serine amide; hydroxyethyl isostearyl isopropanolamine; lactic acid; Lactomide (available from Pentapharm); lanolin; lanolin alcohols; lanosterol; lauric acid N laurylglucamide; lipoic acid; N-acetyl cysteine; N-acetyl-L-serine; N-methyl-L-Serine; Net Sterol-ISO (available from Barnet Products); vitamin B3 compounds (such as niacinamide and nicotinic acid); palmitic acid; panthenol; panthetine; phosphodiesterase inhibitors; PHYTO/CER (available from Intergen); phytoglycolipid millet extract (available from Barnet Products Distributer, located in Englewood, N.J.); PHYTOSPHINGOSINE (available from Gist Brocades, located in King of Prussia, Pa.); PSENDOFILAGGRIN (available from Brooks Industries, located in South Plainfield, N.J.); QUESTAMIDE H (available from Quest); serine; sigmasterol; sitosterol; soybean derived sterols; sphingosine; sphingomylinase; S-lactoyl glutathione; stearic acid; Structurine (available from Silah); SUPER STEROL ESTERS (available from Croda); thioctic acid; THSC CERAMIDE OIL (available from Campo Research); trimethyl glycine; tocopheryl nicotinate; vitamin D₃; Y2 (available from Ocean Pharmaceutical); and mixtures thereof.

Non-steroidal Cosmetic Soothing Actives

Cosmetic soothing actives can be effective in preventing or treating inflammation of the skin and can be included in the controlled release system for stabilizing retinol of the present invention. The soothing active enhances the skin appearance benefits of the present invention, e.g., such agents contribute to a more uniform and acceptable skin tone or color. The exact amount of anti-inflammatory agent to be used in the compositions will depend on the particular anti-inflammatory agent utilized since such agents vary widely in potency. Non-limiting examples of cosmetic soothing agents include the following categories: propionic acid derivatives; acetic acid derivatives; fenamic acid derivatives; biphenylcarboxylic acid derivatives; and oxicams. All of these cosmetic soothing actives are fully described in U.S. Pat. No. 4,985,459 to Sunshine et al., issued Jan. 15, 1991, incorporated by reference herein in its entirety. Non-limiting examples of useful cosmetic soothing actives include acetyl salicylic acid, ibuprofen, naproxen, benoxaprofen, flurbiprofen, fenoprofen, fenbufen, ketoprofen, indoprofen, pirprofen, carprofen, oxaprozin, pranoprofen, miroprofen, tioxaprofen, suprofen, alminoprofen, tiaprofenic acid, fluprofen, bucloxic acid, absinthium, acacia, aescin, alder buckthorn extract, allantoin, aloe, APT (available from Centerchem), arnica, astragalus, astragalus root extract, azulene, Baicalin SR 15 (available from Bamet Products Dist.), baikal skullcap, baizhu, balsam canada, bee pollen, BIOPHYTEX (available from Laboratories Serobiologiques), bisabolol, black cohosh, black cohosh extract blue cohosh, blue cohosh extract, boneset, borage, borage oil, bradykinin antagonists, bromelain, calendula, calendula extract, Canadian Willowbark Extract (available from Fytokem), candelilla wax, Cangzhu, canola phytosterols, capsicum, carboxypeptidase, celery seed, celery stem extract, CENTAURIUM (available from Sederma), centaury extract, chamazulene, chamomile, chamomile extract, chaparral, chaste tree, chaste tree extract, chickweed, chicory root, chicory root extract, chirata, chishao, collodial oatmeal, comfrey, comfrey extract, CROMOIST CM GLUCAN (available from Croda), darutoside, dehurian angelica, devil's claw, divalent metals (such as, magnesium, strontium, and manganese), doggrass, dogwood, Eashave (available from Pentapharm), eleuthero, ELHIBIN (available from Pentapharm), ENTELINE 2 (available from Secma), ephedra, epimedium, esculoside; etbacrynic acid, evening primrose, eyebright, Extract LE-100 (available from Sino Lion), Fangfeng, feverfew, ficin, forsythia fruit, Fytosterol 85 (available from Fytokem), ganoderma, gaoben, Gatuline A (available from Gattefosse), gentian, germanium extract, gingko bilboa extract, ginkgo, ginseng extract, goldenseal, gorgonian extract, gotu kola, grape fruit extract, guaiac wood oil, guggal extract, helenalin esters, henna, honeysuckle flower, horehound extract, horsechestnut, horsetail, huzhang, hypericum, ichthyol, immortelle, ipecac, job's tears, jujube, kola extract, LANACHRYS 28 (available from Lana Tech), lemon oil, lianqiao, licorice root, ligusticum, ligustrum, lovage root, luffa, mace, magnolia flower, manjistha extract, margaspidin, matricin, melatonin, MICROAT IRC (available from Nurture), mints, mistletoe, Modulene (available from Seporga), mono or diglucosides of glabridin, mono or diglucosides of gentisin, MTA (5′-deoxy-5′-methythioadenosine), mung bean extract, musk, N-methyl arginine, oat beta glucan, oat extract, orange, panthenol, papain, phenoxyacetic acid, peony bark, peony root, Phytoplenolin (available from Bio Botanica), phytosphingosine, Preregen (available from Pentapharm), purslane, QUENCH T (available from Centerchem), quillaia, red sage, rehmannia, rhubarb, rosemary, rosmarinic acid, royal jelly, rue, rutin, sandlewood, sanqi, sarsaparilla, saw palmetto, SENSILINE (available from Silab), SIEGESBECKIA (available from Sederma), stearyl glycyrrhetinate, Stimutex (available from Pentapharm), storax, strontium nitrate, sweet birch oil, sweet woodruff, tagetes, tea extract, thyme extract, tienchi ginseng, tocopherol, tocopheryl acetate, triclosan, turmeric, urimei, ursolic acid, white pine bark, witch hazel xinyi, yarrow, yeast extract, yucca, and mixtures thereof.

Artificial Tanning Actives and Accelerators

Artificial tanning actives can help in simulating a natural suntan by increasing melanin in the skin or by producing the appearance of increased melanin in the skin and can be included in the controlled release system for stabilizing retinol of the present invention. Non-limiting examples of artificial tanning agents and accelerators include dihydroxyacetaone; tyrosine; tyrosine esters such as ethyl tyrosinate and glucose tyrosinate; acetyl tyrosine; phospho-DOPA; brazilin; caffeine; coffee extracts; dihydroxyacetone; DNA fragments; isobutyl methyl xanthine; methyl xanthine; Phototan (available from Laboratoires Serobiologiques); prostaglandins; tea extracts; theophylline; tyrosine; UNIPERTAN P2002 and UNIPERTAN P27 (both available from Unichem); and mixtures thereof.

Skin Lightening Actives Skin lightening actives can actually decrease the amount of melanin in the skin or provide such an effect by other mechanisms and can be included in the controlled release system for stabilizing retinol of the present invention. Skin lightening actives suitable for use herein are described in co-pending patent application Ser. No. 08/479,935, filed on Jun. 7, 1995 in the name of Hillebrand, corresponding to PCT Application No. U.S. Ser. No. 95/07432, filed Jun. 12, 1995; and copending patent application Ser. No. 08/390,152, filed on Feb. 24, 1995 in the names of Kalla L. Kvalnes, Mitchell A. DeLong, Barton J. Bradbury, Curtis B. Motley, and John D. Carter, corresponding to PCT Application No. U.S. Ser. No. 95/02809, filed Mar. 1, 1995, published Sep. 8, 1995; all incorporated herein by reference. Non-limiting examples of skin lightening actives useful herein include adapalene, aloe extract, alpha-glycaryl-L-ascorbic acid, aminotyroxine, ammonium lactate, anethole derivatives, apple extract, arbutin, areca catechu L. extract, ascorbic acid, ascorbyl palmitate, azelaic acid, bamboo extract, bearberry extract, bletilla tuber, bupleurum falcatum extract, bumet extract, Bumet Power (available from Barnet Products), butyl hydroxy anisole, butyl hydroxy toluene, butyl resoreinol, Chuanxiong, cola decaballo extract, Dang-Gui, deoxyarbutin, 1,3 diphenyl propane derivatives, 2,5 dihydroxybenzoic acid and its derivatives, 2-(4-acetoxyphenyl)-1,3 dithane, 2-(4-hydroxyphenyl)-1,3 dithane, ellagic acid, escinol, estragole derivatives, esculoside, esculetin, FADEOUT (available from Pentapharm), Fangfeng, fennel extract, gallic acid and its derivatives, ganodenna extract, gaoben, GATULINE WHITENING (available from Gattlefosse), genistic acid and its derivatives, gentisyl alcohol, glabridin and its derivatives, gluco pyranosyl-1-ascorbate, gluconic acid, glucosamine, glycolic acid, glycyrrhizinic acid, green tea extract, 4-Hydroxy-5-methyl-3[2H]-furanone, hydroquinine, 4 hydroxyanisole and its derivatives, 4-hydroxy benzoic acid derivatives, hydroxycaprylic acid, hyptis extract, inositol ascorbate, kojic acid, kojic dipalnitate, lactic acid, lemon extract, licorice extract, Licorice P-TH (available from Barnet Products), linoleic acid, magnesium ascorbyl phosphate, Melfade (available from Pentapharm), MELAWHITE (available from Pentapharm), Melanostatine DM (available from Laboratories Seporga), morus alba extract, mulberry root extract, niacinamide, 5-octanoyl salicylic acid, parsley extract, phellinus linteus extract, pinon blanco extract, pinon negro extract, piri-piri extract, pyrogallol derivatives, retinoic acid, retinol, retinyl esters (acetate, propionate, palmitate, linoleate), 2,4 resorcinol derivatives, 3,5 resorcinol derivatives, rose fruit extract, rucinol, salicylic acid, Song-Yi extract, Sophora Powder (available from Bamet Products), 4-thioresorein, 3,4,5 trihydroxybenzyl derivatives, tranexamic acid, tyrostat (Rumex Extract available from Fytokem), Tyroslat 10,11 (available from Fytokem), vanilla derivatives, vitamin D₃ and its analogs, and mixtures thereof.

Sunscreen Actives

Sun screen agents can be included in the controlled release system for stabilizing retinol of the present invention. The term “sunscreen agent” as used herein defines ultraviolet ray-blocking compounds exhibiting absorption within the wavelength region between about 290 and about 400 nm. Sunscreens can be classified into five groups based upon their chemical structure: para-amino benzoates; salicylates; cinnamates; benzophenones; and miscellaneous chemicals including menthyl anthranilate and digalloyl trioleate. Inorganic sunscreens can also be used including titanium dioxide, zinc oxide, iron oxide and polymer particles such as those of polyethylene, polymethylmethacrylates and polyamides A wide variety of conventional sunscreening agents are suitable for use in the present invention as described in Segarin et al., at Chapter VIII, Pages 189 et seq., “Cosmetics Science and Technology”, the disclosure of which is incorporated herein by reference. Specific suitable sunscreening agents include, for example: p-aminobenzoic acid, its salts and derivatives, anthranilates, salicylates, cinnamic acid derivatives, dihydroxycinnamic acid derivatives, trihydroxycinnamic acid derivatives, hydrocarbons, dibenzalacetone and benzalacetophenone, naphthosulfonates, dihydroxy-naphthoic acid and its salts, o- and p-hydroxy-biphenyldisulfonates, coumarin derivatives, diazoles quinine salts, quinoline derivatives, hydroxy or methoxy substituted benzophenones, uric and vilouric acids, tannic acid and its derivatives, hydroquinone, benzophenones, and the like.

Also useful herein are sunscreening actives. A wide variety of sunscreening agents are described in U.S. Pat. No. 5,087,445, to Haffey et al., issued Feb. 11, 1992; U.S. Patent No. 5,073,372, to Turner et al., issued Dec.17, 1991; U.S. Pat. No. 5,073,371, to Turner et al. issued Dec. 17, 1991; and Segarin, et al., at Chapter VIII, pages 189 et seq., of Cosmetics Science and Technology, all of which are incorporated herein by reference in their entirety. Non-limiting examples of sunscreens which are useful in the compositions of the present invention are those selected from the group consisting of 2-ethylhexyl p-methoxycinnamate, 2-ethylhexyl N,N-dimethyl-p-aminobenzoate, p-aminobenzoic acid, 2-phenylbenzimidazole-5-sulfonic acid, octocrylene, oxybenzone, homomenthyl salicylate, octyl salicylate, 4,4′-methoxy-t-butyldibenzoylmethane, 4-isopropyl dibenzoylmethane, 3-benzylidene camphor, 3-(4-methylbenzylidene) camphor, titanium dioxide, zinc oxide, silica, iron oxide, and mixtures thereof. Still other useful sunscreens are those disclosed in U.S. Pat. No. 4,937,370, to Sabatelli, issued Jun. 26, 1990; and U.S. Pat. No. 4,999,186, to Sabatelli et al., issued Mar. 12, 1991; these two references are incorporated by reference herein in their entirety. Still other useful sunscreens include aminobenzoic acid (PABA), benzylidene camphor, butyl methoxy dibenzoyl methane, diethanolamine p-methoxycinnamate, dioxybenzone, ethyl dihydroxypropyl (PABA), glyceryl aminobenzoate, homomenthyl salicylate, isopropyl dibenzoyl methane, lawsone and dihydroxyacetone, menthyl anthranilate, methyl anthranilate, methyl benzylidene camphor, octocrylene, octyl dimethyl (PABA), octyl methoxycinnamate, oxybenzone, 2-phenylbenzimidazole-5-sulfonic acid, red petrolatum, sulisobenzone, titanium dioxide, triethanolamine salicylate, zinc oxide, and mixtures thereof. Especially preferred examples of these sunscreens include those selected from the group consisting of 4-N,N-(2-ethylhexyl)methylaminobenzoic acid ester of 2,4-dihydroxybenzophenone, 4-N,N-(2-ethylhexyl)methylaminobenzoic acid ester with 4-hydroxydibenzoylmethane, 4-N,N-(2-ethylhexyl)-methylaminobenzoic acid ester of 2-hydroxy-4-(2-hydroxyethoxy)benzophenone, 4-N,N-(2-ethylhexyl)-methylaminobenzoic acid ester of 4-(2-hydroxyethoxy)dibenzoylmethane, and mixtures thereof.

Exact amounts of sunscreens which can be employed will vary depending upon the sunscreen chosen and the desired Sun Protection Factor (SPF) to be achieved. SPF is a commonly used measure of photoprotection of a sunscreen against erythema. See Federal Register, Vol. 43, No. 166, pp. 38206-38269, Aug. 25, 1978, which is incorporated herein by reference in its entirety.

Sebum Stimulators

Sebum stimulators can increase the production of sebum by the sebaceous glands and can be included in the controlled release system for stabilizing retinol of the present invention. These skin care actives are especially useful for post menopausal women who are sebum deficient. Nonlimiting examples of sebum stimulating actives include bryonolic acid, completech MBAC-DS, dehydroetiandrosterone (also known as DHEA), orizanol and mixtures thereof.

Sebum Inhibitors

Sebum inhibitors can decrease the production of sebum by the sebaceous glands and can be included in the controlled release system for stabilizing retinol of the present invention. Non-limiting examples of sebum inhibiting actives include aluminium hydroxy chloride, ASEBIOL (available from Laboratories Serobiologiques), BIODERMINE (available from Sederma), climbazole, COMPLETECH MBAC-OS (available from Lipo), corticosteroids, cucumber extracts, dehydroacetic acid and its salts, dichlorophenyl imidazoldioxolan (available from Elubiol), gugulipiu, ketoconazole, Lichochalcone LR 15 (available from Maruzen), niacinamide, phloretin, PHLOROGINE (available from Secma), Phycosaccharide Anti-Acne (available from Codif), S-carboxyl methyl cysteine, sepicontrol AS, spironolactone, tioxolone, tocopherol, tranexamic acid, UNITRIENOL T27 (available from Unichem), zincidone (UC1B), and mixtures thereof.

Protease Inhibitors

Also useful as active ingredients in the present invention are protease inhibitors. Non-limiting examples of protease inhibitors which are useful in the compositions of the present invention are those selected from the group consisting of A E Complex (available from Barnet Products); ALE (available from Seporga); allicin; alpha lupaline; Aosaine (available from Secma); Aprotinin (available from Pentapharm); areca catechu (Betel Nut) extract; areca catechu extracts; Blue Algae Extract (available from Collaborative Labs); Centaurium (available from Sederma); cholesterol sulfate; CMST (available from Bioetica); Dermoprotectine (available from Sederma); Disacoside HF 60 (available from Barnet Products); Elhibin (available from Pentapharm); Fluid Out Colloid (available from Vegetech); Hypotaurine (available from Sogo Pharmaceutical); In Cyte Heathes (available from Collaborative Labs); Micromerol (available from Collaborative Labs); Pefabloc SP (available from Pentapharm); Sepicontrol AS (available from Seppic); Siegesbeckia (available from Sederma); Sophorine (available from Barnet Products); Thiotaine (available from Barnet Products); uncaria gambis roxburgh extract; zinc and mixtures thereof.

Skin Tightening Agents

Also useful as active ingredients in the present invention are skin tightening agents. Non-limiting examples of skin tightening agents which are useful in the compositions of the present invention are those selected from the group consisting of Biocare SA (available from Amerchol); egg albumen; Flexan 130 (available from National Starch); Gatuline Lifting (available from Gattefosse); Pentacare HP (available from Pentapharm); Vegeseryl (available from Laboratories Serobioloques) and mixtures thereof.

Anti-Itch Ingredients

Also useful as active ingredients in the present invention are anti-itch ingredients. Non-limiting examples of anti-itch ingredients which are useful in the compositions of the present invention are those selected from the group consisting of Stimu-tex (available from Pentapharm); Takanal (available from Ikeda-Distributer); Ichthyol (available from International Sourcing-Distributor); Oxygenated Glyceryl Triesters (available from Seporgia) and mixtures thereof.

Antioxidants

In addition to the retinol the controlled release system of the invention can also contain other antioxidants including those well known in the art. Representative antioxidants include vitamin E, tocopheryl acetate, betaglucan, coenzyme Q10, representative formula CH₃C₆(O)₂(OCH₃)₂)CH₂CH:C(CH₃)CH₂!ₙ H, butylated hydroxy toluene (BHT), butylated hydroxy anisole BHA, superoxide dismutose, propylgallate, and the like.

Skin Conditioners

In addition to the retinol, the controlled release system of the invention can also contain other skin conditioners, moisturizers and surfactants can be included as additives. Illustrative conditioners include mineral oil, petrolatum, vegetable oils (such as soybean or maleated soybean oil), dimethicone, dimethicone copolyol, cationic monomers and polymers (such as guar hydroxypropyl trimonium chloride and distearyl dimethyl ammonium chloride) as well as combinations thereof. Illustrative moisturizers are polyols such as sorbitol, glycerin, propylene glycol, ethylene glycol, polyethylene glycol, polypropylene glycol, 1,3-butane diol, hexylene glycol, isoprene glycol, xylitol, fructose and mixtures thereof.

Drugs

Suitable drugs which can be administered in the controlled release system of the present invention include but are in no way limited to anti-bacterial agents such as thimerosal, chloramine, boric acid, phenol, iodoform, chlorhexidine and other oral antiseptics, beta-lactam antibiotics, for example cefoxitin, n-formamidoyl thienamycin and other thienamycin derivatives, tetracyclines, chloramphenicol, neomycin, gramicidin, kanamycin, amikacin, sismicin and tobramycin; anti-inflammatory steroids such as cortisone, hydrocortisone, beta-methasone, dexamethasone, fluocortolone, prednisolone, triamcinolone and the like; non-steroidal anti-inflammatory drugs including flurbiprofen, ibuprofen, indomethacin, piroxicam, naproxen, antipyrine, phenylbutazone and aspirin; plaque dissolving substances, for example lysozyme chloride or amylase; and local anaesthetics such as lidocaine, procaine, benzocaine, xylocaine and the like. The biologically active ingredient can also be one or more antibiotics, such as penicillin, polymyxin B, vancomycin, kanamycin, erythromycin, niddamycin, metronidazole, spiramycin and tetracycline.

Fragrances, Flavors, and Sensory Markers

The controlled release system of the present invention also provides synchronizing the release of the sensory markers such as fragrances, flavors, cooling agents, such as menthol derivatives, and heating agents, such as capzasin. The release of the sensory markers can be used to convey to the consumer the product performance, provide long lasting odor or flavor perception, and signal that a new application of the product is needed.

The flavoring compositions can be used to enhance existing flavors in, or to provide the entire flavor impression to a foodstuff. Suitable flavoring compositions include organic acids including fatty, saturated, unsaturated and amino acids, alcohols, including primary and secondary alcohols, esters, carbonyl compounds including aldehydes and ketones, lactones, cyclic organic materials including benzene derivatives, alicyclics, heterocyclics such as furans, pyridines, pyrazines and the like, sulfur-containing materials including thiols, sulfides, disulfides and the like, proteins, lipids, carbohydrates, and flavor potentiators such as monosodium glutamate, guanylates, inosinates, natural flavoring materials such as vanillin, and the like. It will be appreciated that the types and amounts of materials selected from the foregoing groups of materials will depend upon the precise organoleptic character desired in the finished product and, especially in the case of flavoring compositions used to enhance other flavors, will vary according to the foodstuff to which the flavor and aroma are to be imparted. Inorganic materials such as sodium chloride and freshness preservers such as butylated hydroxyanisole, butylated hydroxytoluene and propyl gallate can be added for their adjuvant preservative effects on the flavoring composition or on the final food composition itself.

Conventional fragrance ingredients and perfume ingredients can be used in the release system of the present invention. Selection of any perfume component, or amount of perfume, is based on functional and aesthetic considerations. Examples of usable fragrance and flavor compounds discussed hereinafter, along with their odor characters, and their physical and chemical properties, are given in “Perfume and Flavor Chemicals (Aroma Chemicals)”, Steffen Arctander, published by the author, 1969, and in “Common Fragrance and flavor Materials—Preparation, Properties and Uses”, Kurt Bauer and Dorotea Garbe, published by VCH Verlagsgesellschaft mbH, 1985, incorporated herein by reference.

Botanical extracts are oak bark extract, walnut extract, tincture of arnica, hamamelis extract, ribwort extract, pansy extract, thyme or sage extract; for the treatment of damaged or injured skin, for example, St. John's wort tincture, cone flowers tincture, chamomile flowers extract, or calendula flowers tincture; and for the care of exhausted and damaged skin, for example, birch leaves extract, nettle extract, coldsfoot extract, comfrey tincture, horsetail extract, or aloe vera extract. Vegetable preparations may also be released from the film layer for the intradermal treatment of diseases, for example, extracts of horse chestnut and butcher's broom in case of vein diseases, or extracts and tinctures of arnica, calendula, and capsicum in case of contusions, distortions, or haemorrhages. Vegetable preparations in the system according to the present invention may also be used in transdermal therapy, for example, ginseng extract in case of geriatric complaints; valerian tincture, extracts of melissa and hop to cause a sedative effect in case of superexcitation, sleep disturbances, and stress; extracts of kola and tea to achieve a stimulative effect; or hawthorn extract to stabilize the circulatory system.

Preservatives

Preservatives can desirably be incorporated into the controlled release system for stabilizing retinol of the present invention to protect against the growth of potentially harmful microorganisms. While microorganisms tend to grow in the aqueous phase, microorganisms can also reside in the anhydrous or oil phase. As such, preservatives which have solubility in both water and oil are preferably employed in the present compositions. Suitable preservatives for compositions of the present invention are alkyl esters of parahydroxybenzoic acid. Other preservatives, which can be used include hydantoin derivatives, propionate salts, and a variety of quaternary ammonium compounds.

Appropriate preservatives can be selected to satisfy the preservative challenge test and to provide product stability. Particularly preferred preservatives are methylparaben, imidazolidinyl urea, sodium dehydroacetate, propylparaben, trisodium ethylenediamine tetraacetate (EDTA), and benzyl alcohol. The preservative can be selected based on the consideration of possible incompatibilities between the preservative and other ingredients in the release system. Preservatives are preferably employed in amounts ranging from about 0.01% to about 2% by weight of the composition.

Moisturizing Agents

Moisturizing agents, such as glycerol, sodium pyrrolidonecarboxylate, NMFs (normal moisturizing factors) and hyaluronic acid can be used in the release system of the present invention.

Processing Method

The carrier particles of the present invention can be prepared by co-melting retinol and other active ingredients with the matrix materials and then converting the molten mass into particles of the desired size by any of the conventional means for converting melted materials to dry particles or microspheres, such as, by spraying the mass through a nozzle into a cool atmosphere or by drum chilling and grinding. Particle size selection can be accomplished by screening, air stream segregation, and the like.

The process for producing the retinol carrier particles comprises the following stages:

(i) heating the matrix materials, such as solid hydrophobic materials to about 10 degrees above the melting point of the hydrophobic materials, with continuous agitation;

(ii) adding the retinol and other selected active ingredients to the melt with continuous agitation; and

(iii) cooling the melt to ambient temperature to form a dry free-flowing powder composition.

The molten mixture can be converted into a free-flowing powder by spraying processes known in the art, such as spray chilling, spray-congealing, granulation, and the like to create fine or very fine particles, of a substantially spherical shape, having an average particle diameter of from about 0.1 microns to about 500 microns, or more preferably having an average particle diameter of from about 0.5 microns to about 50 microns. The molten mixture can also be converted into a free-flowing powder by drum chilling and grinding.

Spraying processes are particularly suitable in which the melts are converted into fine or very fine particles, primarily of spherical shape, whilst they are finely divided and in free fall. The spraying processes can be assisted by blowing with countercurrent cold air such as by spray-chilling, spray-congealing. Other conventional processes which result in coarse particles are also suitable for producing the retinal carrier particles according to the invention. The processes include, for example, a process in which the melt is discharged on to a cooled roll or cooling belt, and where the mixture is obtained as a pellet in the shape of a drop or as a chip after the melt has solidified, following by grinding the solids to the desired particle size.

A flow agent is preferably added after the powder is manufactured. Flow agents which can be used in the present invention can be silica, clay, starch, and the like which can be added to the particles. Suitable fine silica materials are commercially available as pyrogenic or fumed silica, such as materials sold under Trade names of Cabosil manufactured by G. L. Cabot Inc., Aerogel 500 manufactured by J. M. Huber Corp., Syloid 244, -63, -65 manufactured by W. R. Grace and Co., Li-sil 233 manufactured by Pittsburg Plate Glass Co., and Sipemat D-17 manufactured by Degussa Co. Suitable clay materials include kaolinites and bentonites, as described in British Patent No. 1,460,646.

Spray chilling, or spray congealing is well known in the art and been used commercially in many applications, including foods where the core material is a flavoring oil and cosmetics where the core material is a fragrance oil, see “Flavor Encapsulation”, edited by Risch S. J. and Reineccius G. A., ACS Symposium Series, 1988; “Multiparticulate Oral Drug Delivery” pp. 17-34, edited by Ghebre-Sellassie I., Drugs and the Pharmaceutical Sciences, Vol. 65, 1994 which are incorporated herein as references.

The processing method described herein is simple and economical and is characterized by high loading, reproducibility, versatility, and stability. The method is further illustrated in the non-limiting examples.

Retinol may diffuse from the particles at any of the rates of the following:

(i) at steady-state or zero-order release rate in which there is substantially continuous release per unit of time;

(ii) a first-order release rate in which the rate of release declines toward zero with time; and

(iii) a delayed release in which the initial rate is slow, but then increases with time;

(iv) The active agent contained in the particles can be released an extended period of time up to a period of few days to few weeks, depending on matrix barrier properties, particle size, and active payload.

Particles or microspheres formed of a hydrophobic material provide a controlled release system in order to release the active agent over an extended period of time by molecular diffusion. Active agents in the hydrophobic matrix of the particles can be released by transient diffusion. The theoretical early and late time approximation of the release rate of the retinol and other active ingredients dissolved in the hydrophobic matrix of the particles can be calculated from the following equations:

Early time approximation

(mₜ/m_{sec})<0.4

$$\begin{matrix}\frac{{M}_{t}}{{M}_{∞}} = 4 {( \frac{{D}_{p} t}{Π {r}^{2}} )}^{1 / 2} - \frac{{D}_{p} t}{{r}^{2}} & ( 1 ) \\ \frac{ⅆ {M}_{t} / {M}_{∞}}{ⅆ t} = 2 {( \frac{{D}_{p}}{Π {r}^{2} t} )}^{1 / 2} - \frac{{D}_{p}}{{r}^{2}} & ( 2 )\end{matrix}$$

Late Time Approximation

$$\begin{matrix}( {m}_{t} / {m}_{∞} ) > 0.6 \\ & \\ \frac{{M}_{t}}{{M}_{∞}} = 1 - \frac{4}{{( 2.405 )}^{2}} exp ( \frac{- {( 2.405 )}^{2} {D}_{p} t}{{r}^{2}} ) & ( 3 ) \\ \frac{ⅆ {M}_{t} / {M}_{∞}}{ⅆ t} = 1 - \frac{4 {D}_{p}}{{r}^{2}} exp ( \frac{- {( 2.405 )}^{2} {D}_{p} t}{{r}^{2}} ) & ( 4 )\end{matrix}$$

wherein:

r is the radius of the cylinder,

m_{∞} is the amount fragrance released from the controlled release system after infinite time;

mₜ is the amount fragrance released from the controlled release system after time t; and

Dₚ is the diffusion coefficient of the fragrance or aroma chemical in the matrix.

The invention can be further illustrated by the following examples preferred embodiments thereof, although it will be understood that these examples are included merely for purposes of illustration and are not intended to limit the scope of the invention unless otherwise specifically indicated. All percentages, ratios, and parts herein, in the Specification, Examples, and claims, are by weight and are approximations unless otherwise stated.

### Preparation of Stabilized Retinol

### EXAMPLE 1

The following procedure is used for the preparation of stabilized retinol in the hydrophobic particles of the present invention. The solid hydrophobic particles are composed of glyceryl monostearate, commercially available from Jeen International of Fairfield N.J. under the trade name Jeechem GMS-450. Retinol was obtained from BASF as a 50% solution in polysorbate 20, under the name Retinol 50C. 198 grams of glyceryl monostearate, commercially available from Jeen International of Fairfield N.J. under the trade name Jeechem GMS-450.are placed in the oven at 80 degree C. 4 grams of retinol (retinol 50C commercially available from BASF) are added to the melt while mixing it with a glass rod. This molten solution is atomized into a chamber with ambient temperature air passing through the chamber. The atomized droplets freeze into solid particles in the size range of about 5 microns to about 50 microns.

### EXAMPLE 2

The following procedure is used for the preparation of stabilized retinol in the hydrophobic particles of the present invention. The solid hydrophobic particles are composed of glyceryl monostearate, commercially available from Jeen International of Fairfield N.J. under the trade name Jeechem GMS-450. Retinol was obtained from BASF as a 50% solution in polysorbate 20, under the name Retinol 50C. 196 grams of glyceryl monostearate, commercially available from Jeen International of Fairfield N.J. under the trade name Jeechem GMS-450.are placed in the oven at 80 degree C. 4 grams of retinol (retinol 50C commercially available from BASF) are added to the melt while mixing it with a glass rod. This molten solution is atomized into a chamber with ambient temperature air passing through the chamber. The atomized droplets freeze into solid particles in the size range of about 5 microns to about 50 microns.

### EXAMPLE 3

100 grams of glyceryl monostearate (commercially available from Jeen International of Fairfield N.J. under the trade name Jeechem GMS-450) and 96 grams of Purester 34 (Purester is a vegetable derived esters produced from naturally derived fatty alcohol & methyl ester feedstocks which are non-GMO vegetable based renewable resources, commercially available from Strahl & Pitsch Inc. of West Babylon, N.Y.) are placed in the oven at 70 degree C. 4 grams of retinol (retinol 50C commercially available from BASF) are added to the melt while mixing it with a propeller mixer. This molten solution is cooled at room temperature in an aluminum tray. The waxy film layer is cooled by liquid nitrogen and ground with a coffee grinder. The fine powder is sieved into solid particles in the size range of 50 to about 100 microns.

### EXAMPLE 4

The following procedure is used for the preparation of stabilized retinol in the hydrophobic particles of the present invention. The solid hydrophobic particles are composed of glyceryl monostearate (commercially available from Jeen International of Fairfield N.J. under the trade name Jeechem GMS-450) and alkylated polyvinylpyrrolidines, Ganex® V-220 copolymer commercially available from the ISP Company.

100 grams of glyceryl monostearate (commercially available from Jeen International of Fairfield N.J. under the trade name Jeechem GMS-450) and 96 grams of alkylated polyvinylpyrrolidines, Ganex® V-220 copolymer, commercially available from the ISP Company, are placed in the oven at 70 degree C. 4 grams of retinol (retinol 50C commercially available from BASF) are added to the melt while mixing it with a propeller mixer. This molten solution is atomized into a chamber with ambient temperature air passing through the chamber. The atomized droplets freeze into solid particles in the size range of about 5 microns to about 50 microns.

### EXAMPLE 5

The following procedure is used for the preparation of stabilized retinol in the hydrophobic particles of the present invention. The solid hydrophobic particles are composed of alkylated polyvinylpyrrolidines, Ganex® WP-660 copolymer, commercially available from the ISP Company

960 grams of Ganex® WP-660 copolymer, commercially available from the ISP Company, are placed in the oven at 70 degree C. 40 grams of retinol (retinol 50C) commercially available from BASF) are added to the melt while mixing it with a propeller mixer. This molten solution is atomized into a chamber with ambient temperature air passing through the chamber. The atomized droplets freeze into solid particles in the size range of about 5 microns to about 50 microns.

### Test Methods

### EXAMPLE 6

The stability of retinal in the micro-sphere and in the product was evaluated using HPLC by the procedure provided by BASF. Solution of retinal in isopropyl alcohol/n-heptan are submitted to straight phase chromatography with UV detection (325 nm). Evaluation is accomplished by the area percent method. 80 mg of sample are weighted into a volumetric flask. After dissolution in eluent aliquots are applied to the HPLC system.

Chromatographic Conditions:

Column: stainless steel column (250×3 mm) packed with Nucleosil SI 50-5

Eluent: n-heptane; isopropyl alcohol 1000:3 (V/V)

Flow rate: 0.9 m/min

Injection volume: 10 μm

Column temperature: ambient

Detection: UV 325 nm

### Incorporation of the Hydrophobic Particles Comprising the Stabilized Retinol in Cosmetic Products

### EXAMPLE 7

The stabilized retinal of examples 1 to 5 were incorporated in following face cream formulation (BASF formulation, Happi Magazine Formulary section, August 2000 issue):

| Ingredients: | % Wt. |
|---|---|
| Phase A | |
| Cremophor A 25 (BASF) (ceteareth-25) | 2.00 |
| Cremophor A 6 (BASF) (ceteareth-6 (and) | 2.00 |
| stearyl alcohol) | |
| Jojoba oil | 3.00 |
| Cetearyl alcohol | 3.00 |
| Mineral oil | 10.00 |
| Petrolatum | 5.00 |
| Caprylic/capric triglyceride | 4.00 |
| Phase B | |
| Propylene glycol | 5.00 |
| Disodium EDTA | 0.10 |
| Preservative | q.s. |
| Carbomer | 0.20 |
| Deionized water | 64.02 |
| Phase C | |
| Sodium hydroxide | 0.08 |
| Phase D | |
| BHT | 0.10 |
| Vitamin E Acetate (BASF) (tocopherol | 0.50 |
| acetate) | |
| Neat or Encapsulated Retinol | 1.00 |
| Perfume | q.s. |

Procedure:

Heat phases A and B separately to about 80° C. Stir phase B into phase A while homogenizing. Stir in phase C and re-homogenize. Cool to about 40° C., add phase D and homogenize again. Production of the emulsion and the filling into appropriate containers should be done in the absence of oxygen. Viscosity: 10000 mPas Brookfield RVD VII+; pH: 6.5

The stability of the neat and encapsulated retinal in the above product was evaluated at 45° C. for four weeks and the results are shown in table 1:

Table 1: Comparison of the Stability of Neat and Encapsulated Retinal Using the Method of Our Invention

| | Percentage of Stable | | |
|---|---|---|---|
| | Retinol of Initial Amount | | |
| Sample Number | Week 1 | Week 2 | Week 4 |
| Neat (50C) | 55% | 40% | 22% |
| Example 1 | 89% | 63% | 30% |
| Example 2 | 90% | 75% | 50% |
| Example 3 | 75% | 52% | 35% |
| Example 4 | 85% | 65% | 40% |
| Example 5 | 91% | 72% | 48% |

The results clearly show that the controlled release system of the present invention enhances the stability of retinol.

It is to be understood that the above-described embodiments are illustrative of only a few of the many possible specific embodiments which can represent applications of the principles of the invention. Numerous and varied other arrangements can be readily devised in accordance with these principles by those skilled in the art without departing from the spirit and scope of the invention.

What is claimed is:

1. An retinol composition comprising:
a plurality of particles or microspheres having an effective amount of retinol encapsulated in said particle or microsphere and said particle or said microsphere is formed of a hydrophobic matrix material.

2. The composition of claim 1 wherein said hydrophobic material is selected from one or more of the group consisting of natural wax, synthetic wax, vegetable wax, natural wax and silicon copolymer, synthetic wax and silicon copolymer, fatty acid esters, fatty alcohols, solid hydrogenated plant oil, natural polymers and synthetic polymers.

3. The composition of claim 1 wherein said hydrophobic material is selected from the group consisting of alkylated polyvinyl pyrolidine, hydrogenated castor oil, hydrogenated vegetable oil, hard paraffin, glyceral monstearate, hard fat, triglyceride and mixtures thereof.

4. The composition according to claim 1 wherein said hydrophobic material is alkylated polyvinyl pyrrolidone.

5. The composition according to claim 1 wherein said hydrophobic material is glycerol monostearate.

6. The composition of claim 1 further comprising an active agent comprising one or more of a cosmetic agent, dermatological agent or pharmaceutical agent.

7. The composition of claim 1 further comprising an active agent comprising one or more agents selected from the group consisting of: anti-oxidant; free radical scavenger; moisturizer; depigmentation agent; reflectant; humectant; antimicrobial agent; antibacterial agent; allergy inhibitor; anti-acne agent; anti-aging agent; anti-wrinkling agent, antiseptic; analgesic; anti-hair loss agent; hair growth promoting agent; hair growth inhibitor agent; keratolytic agent; anti-inflammatory agent; freshener; healing agent; anti infective agent; inflammation inhibitor; vasoconstrictor; vasodilator; wound healing promoter; peptide, polypeptide; protein; deodorant; antiperspirant; skin emollient; skin moisturizer; hair conditioner; hair softener; hair moisturizer; tanning agent; skin lightening agent; antifungal; depilating agent; counterirritant; poison ivy agent; poison oak agent; burn product; make-up preparation; vitamin; amino acid and their derivatives; herbal extract; flavoid; cooling agent; heating agent; skin conditioner; chelating agent; cell turnover enhancer; coloring agent; sunscreen; nourishing agent; moisture absorber; sebum absorber; and skin penetration enhancer.

8. The composition of claim 1 further comprising an active agent comprising an antimicrobial agent or an antifungal agent.

9. The composition of claim 1 further comprising an active agent comprising one or more antiinflammatory agents selected from the group consisting of: hydrocortisone; oxican, salicylate, acetic acid derivative, fanamate, propionic acid derivative, pyrazole, substituted phenyl compound, 2-naphthyl containing compound, natural anti-inflammatories, and aloe vera.

10. The composition of claim 1 further comprising an active agent comprising a skin barrier repair agent.

12. The composition of claim 11 wherein said skin barrier repair agent is selected from the group consisting of: ascorbic acid; biotin; biotin ester; brassicasterol; caffeine; campesterol; canola derived sterols; cholesterol; cholesterol hydroxystearate; cholesterol isostearate; galactocerebroside; glyceryl serine amide; hydroxyethyl isostearyl isopropanolamine; lactic acid; lanolin; lanolin alcohol; lanosterol; lauric acid N laurylglucamide; lipoic acid; N-acetyl cysteine; N-acetyl-L-serine; N-methyl-L-Serine; vitamin B3 compounds; niacinamide; nicotinic acid; palmitic acid; panthenol; panthetine; phosphodiesterase inhibitors; phytoglycolipid millet extract; serine; sigmasterol; sitosterol; soybean derived sterols; sphingosine; sphingomylinase; S-lactoyl glutathione; stearic acid; thioctic acid; trimethyl glycine; tocopheryl nicotinate; vitamin D₃; and mixtures thereof.

12. The composition of claim 1 further comprising an active agent comprising a non-steroidal cosmetic soothing agent.

13. The composition of claim 12 wherein said non-steroidal cosmetic soothing agent selected from the group consisting of propionic acid derivatives; acetic acid derivatives; fenamic acid derivatives; biphenylcarboxylic acid derivatives; and oxicams.

14. The composition of claim 1 further comprising an active agent comprising a protease inhibitor agent.

15. The composition of claim 14 wherein the protease inhibitor comprises allicin; alpha lupaline; areca catechu; Blue Algae Extract; cholesterol sulfate; uncaria gambis roxburgh extract; zinc and mixtures thereof.

16. The composition of claim 1 further comprising an active agent comprising a skin tightening agent.

17. The composition of claim 1 further comprising an active agent comprising an anti-itch agent.

18. The composition of claim 1 further comprising an active agent comprising an antioxidant.

19. The composition of claim 1 further comprising an active agent comprising a skin conditioner or skin moisturizer.

20. The composition of claim 1 further comprising an active agent selected from the group consisting of mineral oil, petrolatum, vegetable oil, dimethicone, dimethicone copolyol, cationic monomers, cationic polymers, guar hyroxypropyl trimonium chloride, distearyl dimethyl ammonium chloride, sorbitol, glycerin, propylene glycol, ethylene glycol, polyethylene glycol, polypropylene glycol, 1,3-butane diol, hexylene glycol, isoprene glycol, xylitol, fructose and mixtures thereof.

21. The composition of claim 1 further comprising an active agent comprising an anti-bacterial agent selected from the group consisting of thimerosal, chloramine, boric acid, phenol, iodoform, chlorhexidine, oral antiseptics, beta-lactam antibiotics, cefoxitin, n-formamidoyl thienamycin, thienamycin derivatives, tetracyclines, chloramphenicol, neomycin, gramicidin, kanamycin, amikacin, sismicin, tobramycin and combinations thereof.

22. The composition of claim 1 further comprising an active agent comprising an anti-inflammatory selected from the group consisting of cortisone, hydrocortisone, beta-methasone, dexamethasone, fluocortolone, prednisolone, triamcinolone, flurbiprofen, ibuprofen, indomethacin, piroxicam, naproxen, antipyrine, phenylbutazone; aspirin and combinations thereof.

23. The composition of claim 1 further comprising an active agent comprising a local anaesthetic selected from the group consisting of lidocaine, procaine, benzocaine, xylocaine and mixtures thereof.

24. The composition of claim 1 further comprising an active agent comprising a biologically active ingredient selected from the group consisting of antibiotics, penicillin, polymyxin B, vancomycin, kanamycin, erythromycin, niddamycin, metronidazole, spiramycin, tetracycline and combinations thereof.

25. The composition of claim 1 further comprising an active agent comprising a sensory member selected from the group consisting of fragrances, flavors, cooling agents and heating agents.

26. The composition of claim 1 further comprising an active agent comprising a preservative.

27. The composition of claim 26 wherein the preservative is selected from the group consisting of alkyl esters of parahydroxybenzoic acid, hydantoin derivatives, propionate salts, and quaternary ammonium compounds.

28. The composition of claim 1 further comprising an active agent comprising moisturizing agents selected from the group consisting of glycerol, sodium pyrrolidonecarboxylate and hyaluronic acid.

29. The composition of claim 1 wherein said retinol is present in an amount from about 0.01% to about 50% by weight of the composition.

30. The composition of claim 1 wherein said particle or micro-sphere has a diameter of from about 0.1 to about 500 microns.

31. The composition according to claim 1 wherein each of said nano-spheres has an average size of about 0.5 to about 50 microns.

32. The composition according to claim 1 wherein said retinol is released continuously for an extended period of time.

33. The composition according to claim 30 wherein the extended period of time is in the range of a day to a period of a few weeks.

34. The composition of claim 1 wherein the retinol is selected from the group consisting of retinols, retinol derivatives, extracts containing retinol, retinyl esters, retinoic acid and mixtures thereof.

35. The composition of claim 1 wherein said retinol is absorbed on an oil absorbing material prior to being encapsulated in said particle.

36. An article of manufacturer comprising the composition of claim 1.

37. The article of manufacturer of claim 36 wherein the article is selected from the group consisting of a liquid, powder, gel, lotion, cream, spray, stick, ointment and paste.

38. A method for forming the composition of claim 1 comprising the steps of:
heating one or more hydrophobic materials to a temperature above the melting point of the materials to form a melt;
adding retinol into the melt; and
cooling the melt to ambient temperature to form a dry free flowing composition.

39. The method of claim 38 wherein before said step of adding retinol to the melt further comprising the step of:
absorbing retinol on an oil absorbing material.
